(19) United States
(12) Patent Application Publication
(10) Pub. No.: US 2014/0088454 A1
(43) Pub. Date: Mar. 27, 2014

(54) ADHESIVE SHOCK PATCH

(71) Applicant: X2 Biosystems, Inc., Seattle, WA (US)

(72) Inventor: Christoph Mack, Seattle, WA (US)

(73) Assignee: X2 Biosystems, Inc., Seattle, WA (US)

(21) Appl. No.: 14/040,111

(22) Filed: Sep. 27, 2013

Related U.S. Application Data
(60) Provisional application No. 61/706,490, filed on Sep. 27, 2012.

(51) Int. Cl. A61B 5/00

(52) U.S. Cl. CPC A61B 5/6833
USPC 600/553

(57) ABSTRACT

A shock patch is configured for use with a human being to detect various parameters related to the condition of the portion of the human being to which the shock patch is adhered. The patch may be attached to a head using an adhesive sticker. The patch includes sensors for detecting movement of the head, together with memory, processing, and other features to interpret the movement and provide information about the motion or other qualities of the head to which the patch is attached.

### PRIORITY CLAIM

This application claims the benefit of U.S. Provisional Application Ser. No. 61/706,490 filed Sep. 27, 2012, the contents of which are hereby incorporated by reference.

### BACKGROUND OF THE INVENTION

Participation in athletic activities often exposes the participants to a risk of physical harm as a result of such participation. In some cases, the physical harm includes the potential for head injuries, particularly in athletic events where collisions between participants frequently occur (e.g., football, field hockey, lacrosse, ice hockey, soccer and the like). In connection with such sports where deliberate collisions between participants occur, the potential for concussions or other head injuries is greatly enhanced. Although most concussions occur in high-impact sports, athletes in low-impact sports are not immune to mild traumatic brain injury. Head injuries are caused by positive and negative acceleration forces experienced by the brain and may result from linear or rotational accelerations (or both). Both linear and rotational accelerations are likely to be encountered by the head at impact, damaging neural and vascular elements of the brain.

At the school level, school authorities have become sensitive to the risk of injury to which student participants are exposed, as well as to the liability of the school system when injury results. Greater emphasis is being placed on proper training and instruction to limit potential injuries. Some players engage in reckless behavior on the athletic field or do not appreciate the dangers to which they and others are subject by certain types of impacts experienced in these athletic endeavors. Unfortunately, the use of mouth guards and helmets does not prevent all injuries. One particularly troublesome problem is when a student athlete experiences a head injury, such as a concussion, of undetermined severity even when wearing protective headgear. Physicians, trainers, and coaches utilize standard neurological examinations and cognitive questioning to determine the relative severity of the impact and its effect on the athlete. Return to play decisions can be strongly influenced by parents and coaches who want a star player back on the field.

The same problem arises in professional sports where the stakes are much higher for a team, where such a team loses a valuable player due to the possibility of a severe head injury. Recent medical data suggests that lateral and rotational forces applied to the head and neck area (for example, flexion/extension, lateral flexion, and axial rotation) are more responsible for axonal nerve damage than previously thought. Previous medical research had indicated that axially directed forces (such as spinal compression forces) were primarily responsible for such injuries.

Identifying the magnitude of acceleration that causes brain injury may assist in prevention, diagnosis, and return-to-play decisions. Most field measurements assess the acceleration experienced by the player with accelerometers attached to the helmet. In some instances sensors have been placed in mouth guards or in helmets in an effort to detect when an individual has experienced an event that may be associated with injury. Such prior efforts have a variety of drawbacks and are not readily suitable to a wide range of activities.

### SUMMARY OF THE INVENTION

The present invention relates to a shock patch, preferably including an adhesive shock patch, for use with a human being to detect various parameters related to the condition of the portion of the human being to which the shock patch is adhered. By way of example, a preferred patch would include sensors for detecting movement of the user's head, together with memory, processing, and other features to interpret the movement and provide information about the motion or other qualities of the head to which the patch is attached.

In accordance with various preferred embodiments of the invention, the patch electronics module may be removably attached to an adhesive sticker.

In some versions of the invention, the adhesive sticker is formed to have a larger footprint than the footprint of the electronics module.

In a preferred version, the adhesive sticker has a generally triangular, or pear shape.

In further preferred examples, the electronics module includes a pair of electrodes and the sticker includes a corresponding pair of openings, such that the electrodes extend through the openings for direct contact with a wearer's skin.

In other versions of the invention, the patch is adhered to a person's head by a headband. In a preferred implementation of the headband version of the invention, the electronics module is removably adhered to a portion of the headband.

In a preferred headband example, the electronics module includes a pair of electrodes and the headband includes a corresponding pair of openings, such that the electrodes extend through the openings for direct contact with a wearer's skin.

These and other preferred versions of the invention are described in greater detail below.

### BRIEF DESCRIPTION OF THE DRAWINGS

Preferred and alternative examples of the present invention are described in detail below with reference to the following drawings.

FIG. 1 is an illustration of a person applying a preferred adhesive shock patch at a preferred location on a head.

FIG. 2 is a block diagram of a preferred implementation of an adhesive shock patch.

FIG. 3 is an exploded view of a shock patch electronics module and a corresponding preferred adhesive sticker.

FIG. 4 is a plan view of a preferred adhesive sticker.

FIG. 5 is a top plan view of a preferred patch electronics module.

FIG. 6 is a bottom plan view of a preferred patch electronics module.

FIG. 7 is a plan view of an alternate preferred adhesive sticker.

FIG. 8 is an illustration of a headband version of an adhesive patch applied to a person's head.

FIG. 9 is an exploded view of a shock patch electronics module and a corresponding headband.

### DETAILED DESCRIPTION OF THE PREFERRED EMBODIMENT

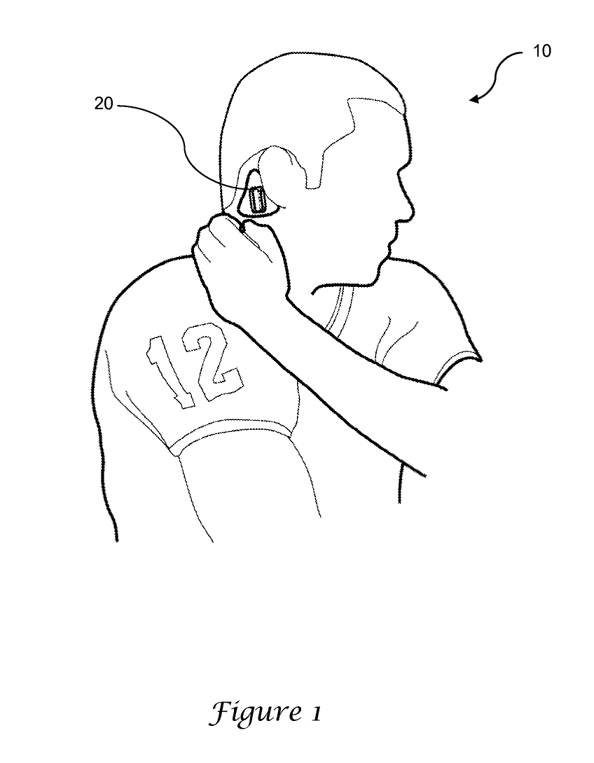

As illustrated in FIG. 1, a preferred shock patch 20 may be attached to the head of the human 10. In the preferred version as illustrated, the patch 20 is adhered behind the ear. Other locations on the head may also be used, such as on the temples, cheekbones, bridge of the nose, or other positions on the head. While FIG. 1 illustrates a single patch 20, in accordance with this invention two or more patches may also be used simultaneously.

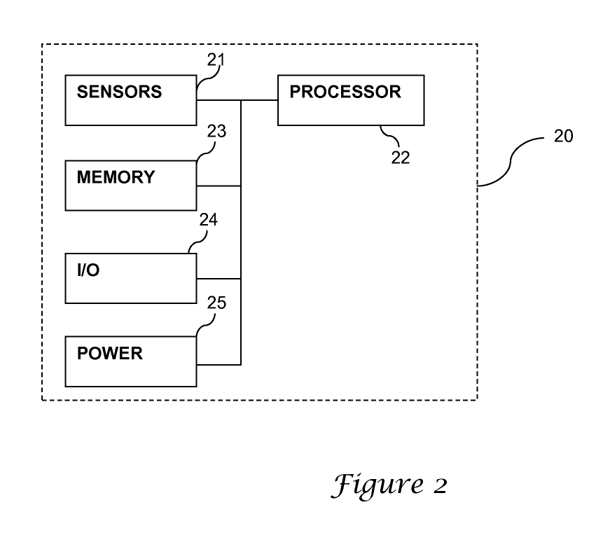

With reference to the block diagram of FIG. 2, the preferred patch 20 includes at least one sensor 21, a processor 22, memory 23, an I/O means 24 for input and/or output to or from the patch, and a battery or power supply 25. In one version, the patch is configured with one or more impact sensors. The impact sensors preferably comprise a plurality of low-cost distributed impact sensors arranged on the patch to detect acceleration in three axes. Most preferably, the sensors are each in the form of a linear accelerometer able to detect acceleration in three axes in addition to an angular rate sensor able to detect angular rate in three axes. Other sensors such as a magnetometer able to detect angular displacement may also be used.

Any of a variety or electronic devices may be used to monitor the patch (and therefore the wearer's head) for impact or acceleration events. For example, the sensors may be MEMS type impact sensors, MEMS accelerometers, miniature weighted cantilevers fitted with miniature strain-gauge elements, piezoelectric membranes, or Force-Sensitive-Resistors (FSR). The sensors may also include one or more gyroscopes positioned to detect acceleration along one or more axes.

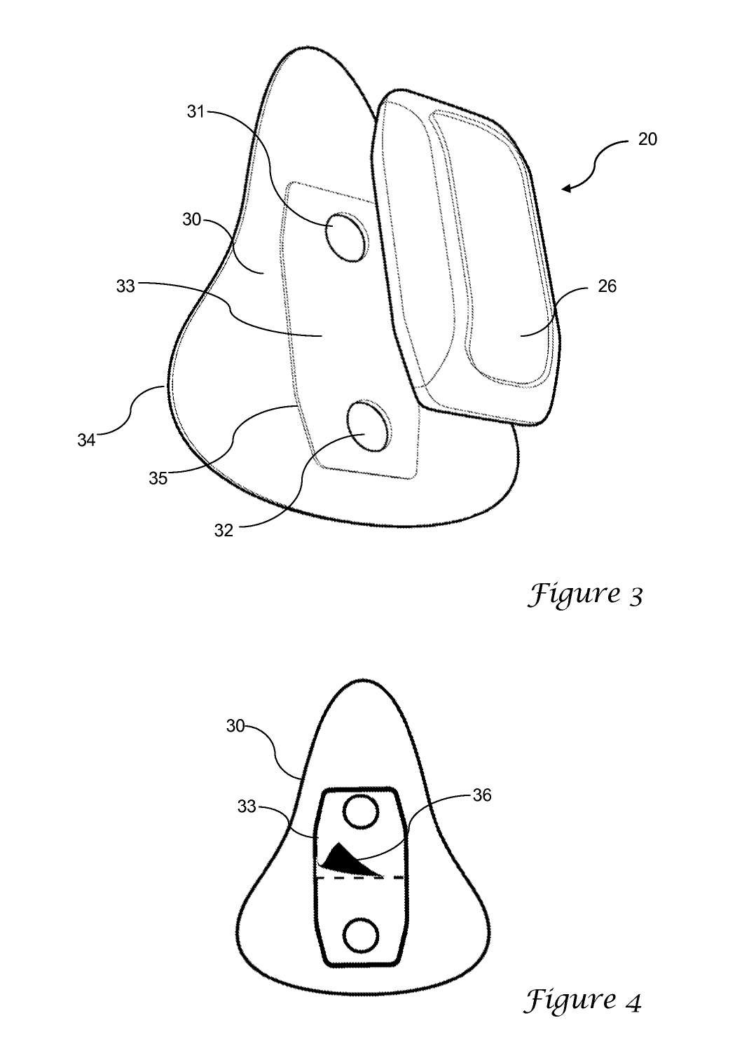

The sensors are secured to the patch and preferably encased within a protective covering that will allow the sensors to be securely mounted to the patch and protected from damage by direct contact. With reference to FIG. 3, the patch 20 includes an electronics module containing the components described with reference to FIG. 2, with the electronics module preferably housed in an outer casing 26.

In a preferred example of the invention, the casing is formed from a rigid plastic material such as acrylic, PETG, PVC, or polycarbonate in order to provide sturdy protection for the electronics components contained inside the casing. This form of the invention having a rigid casing may be particularly preferred for use in high contact sports, to ensure that the electronics are securely protected and the shock patch continues to function. In one version, an outer surface of the patch casing comprises an elastomeric material to provide a cushioning effect. The elastomeric material may be applied outside the rigid plastic casing or, alternatively, may be used as a casing without the use of a separate rigid casing.

The casing 26 may take any shape, but in accordance with one preferred version the casing has an elongated shape in which a length is greater than a width. Thus, in the version as illustrated FIG. 3, the casing 26 includes a vertical length (as seen on the page) that is greater than the horizontal width. This elongated shape serves to naturally guide application of the patch in a particular orientation, aiding in determining the axial reference frame of the patch on the user. In other versions, the patch may employ other visual, physical, or electronic means for determining an axial frame of reference.

In one version of the invention, one side of the casing 26 may contain an adhesive that is formulated to stick to the casing and the skin of a person. The adhesive may be applied to the casing prior to each use for better adhesion and to allow the patch to be re-used. Alternatively, the adhesive may be applied at the time of manufacture, particularly in the case of a patch that is intended to be disposable and for single use only.

As best seen in FIG. 3, a preferred adhesive shock patch and outer casing 26 is produced such that it is separated from an adhesive sticker 30. In this configuration, the sticker 30 may be discarded after use so that the patch 20 and outer casing 26 may be re-used. In a preferred version incorporating a separate sticker 30 having a perimeter 34, the sticker includes an interior casing adhesion area 33 having a casing adhesion perimeter 35 in which the casing adhesion perimeter 35 matches the footprint of the casing 26 when the casing is attached to the sticker. The casing adhesion area 33 further includes a pair of holes 31, 32 passing through the sticker 30 to receive a corresponding pair of electrodes or other sensors provided on the casing, as described further below.

With reference to FIG. 4, the sticker 30 includes a backing sheet 36 that covers the adhesive of the casing adhesive area 33 until the sticker is ready for use. When a user is ready to use a patch, the backing sheet 36 is removed to expose the adhesive and the lower side of a casing 26 is attached to the exposed adhesive of the casing adhesion area 33. The casing adhesion area is positioned on a front side of the sticker 30, as is visible in FIGS. 3 and 4. An opposing back side of the sticker (not visible in FIGS. 3 and 4) likewise includes a backing sheet covering an adhesive. In order to attach the patch and sticker to a person's head (such as shown in FIG. 1), the backing sheet is removed and the back side of the sticker is attached to the skin. This configuration likewise attaches the patch and casing to the wearer, as shown in FIG. 1, because the casing is adhered to the sticker, which in turn is adhered to the skin.

In alternate versions, the sticker includes an adhesive back side as described above, but incorporates a hook and loop fastener for attaching the patch 20 to the sticker 30. Thus, the front side of the sticker includes a first component of a hook and loop fastener while the back side of the casing includes the second complementary component of a hook and loop fastener, thereby allowing the patch 20 to be removably attached to the sticker.

The sticker 30 is preferably formed in a generally triangular shape, as best seen in FIG. 4. As also seen in FIG. 4, the footprint of the patch casing adhesive area 33 is smaller than that of the sticker 30, providing a portion of the sticker extending beyond the footprint of the casing around the entire boundary of the casing. By extending the sticker beyond the casing, the adhesion is stronger and a partial separation of the sticker from the skin is less likely to result in the casing and patch falling off.

It is useful for the electronics system within the patch 20 to have a positional and axial frame of reference, and the evaluation of any potential impact events are best performed with an understanding of the orientation of the patch and the positioning on the wearer. A preferred patch may optionally include a small orientation sensor positioned on or within the patch to determine and orientation of the patch. Low-cost, small MEMS orientation sensors are available and sufficiently sized to be incorporated into a preferred patch to provide information to the processor regarding the positional orientation of the patch. The patch would preferably also include a visual indicator providing information to the wearer regarding a preferred orientation for the patch when applied to the wearer.

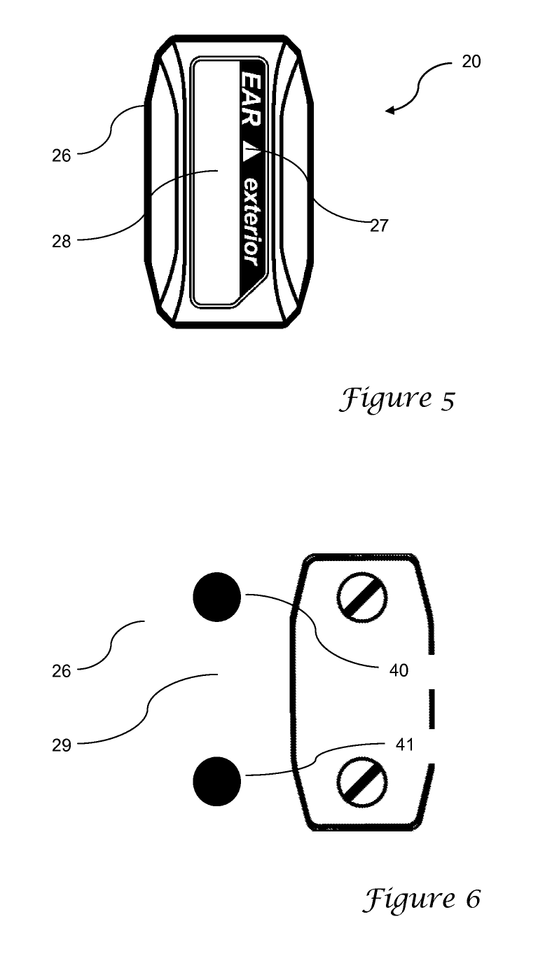

In one version of the invention, as shown in FIG. 5, the casing 26 of the patch 20 may include a reference frame marking 27 or indicator pointing in a particular direction to aid in positioning the patch in a particular way. In the illustrated version, the reference frame marking comprises an arrow and the word “EAR” such that the arrow points to the desired direction of the person's ear when the patch is properly attached. In one version the patch may include a further external indicator pointing to up or down along with an indicator pointing toward an ear, such that application of the patch in accordance with the indicators ensures that the data can be linked to a particular location on a person's head.

Alternatively, the patch may include a sensor to determine the up and down positions, or include an algorithm stored in memory that interprets inputs from the sensors in order to determine which direction is up and which direction is down. In one version, the up/down orientation of the patch may be determined by using accelerometer data collected shortly after the patch is applied. It would be expected that, particularly in the first few seconds after the device is applied, the accelerometers will detect the gravity vector data but little or no acceleration of the head in other directions. The software on board the patch is programmed to evaluate the accelerometer data during this initial period in order to determine the direction most likely associated with up or down. This determined orientation can then be used later in evaluating the subsequent acceleration events to better determine a particular vector of acceleration with respect to the wearer's head.

In such a configuration, the indicator 27 together with the determined up and down direction enable the processor (or a remote processing system analyzing the data) to determine whether the patch was positioned behind the left ear or the right ear. For example, if “up” is determined to be at the end of the casing adjacent the word “EAR” in FIG. 5, then the patch must have been placed behind the right ear if it is aligned as indicated, with the arrow pointing to the closest ear. But if “up” is determined to be at the end of the word “exterior” (that is, opposite the word EAR) then the patch must have been placed behind the left ear if it is aligned as indicated with the arrow properly pointing toward the adjacent ear. Thus, the combination of the external indicator and additional processing enables the patch or an external processing system to determine an axial frame of reference around the patch and to understand the location of the patch, behind either the left or right ear.

The preferred patch may optionally include one or more proximity sensors or other such sensors to determine whether the patch is adhered to a person. In a preferred version, two sensors are provided, positioned on the bottom side of the patch. With reference to FIG. 5, a top side 28 of the casing 26 is shown. The corresponding opposing bottom side 29 of the same patch 20 and patch casing 26 is shown in FIG. 6. Two sensors 40, 41 protrude out of the bottom side of the casing. In a preferred version, each sensor is in the form of a short metal post extending just beyond the surface of the bottom side of the casing. Though two such posts are shown, depending on the nature of the sensors used for proximity a greater or lesser number may be used.

Functionally, a primary purpose of the proximity sensor is to determine whether the patch is in position and worn by the user. Thus, the proximity sensors may be placed in any location that would allow the sensors to determine that the patch is applied to a person. The proximity sensor may take any form so long as it is able to determine whether the patch is applied. As one preferred example, the proximity sensor is a capacitive sensor. Capacitive sensors are commonly employed in touch screen computer displays and generally operate to detect the presence of anything that is conductive or which has dielectric properties. Capacitive sensors can be employed with a hard surface material such as is used with touch-screen displays, though the use of such a material may be less ideal when incorporated into a patch. In one version, the capacitive sensor is incorporated into a flexible material which is then used as a portion of the patch such that the capacitive sensor will be in contact with the player's head when the patch is worn by the user.

As described above, in a preferred version at least two proximity sensors are used. Where multiple sensors are provided, the system polls each of the proximity sensors to determine whether all or a majority of the proximity sensors detect the presence of a capacitive object such as the wearer's head. If so, then the system determines that any impact events detected by the impact event sensors are related actual events experienced by the head of the wearer as opposed to spurious events experienced by the patch alone.

In another version of the invention, the proximity sensors may comprise a pair of electrodes that form an open circuit when the patch is not in contact with human skin, but which form a short or closed-circuit when the patch is applied. Thus, as illustrated in FIG. 6, two such electrodes would be prominent and exposed on the bottom side of the patch for this purpose.

Particularly where contact with the skin is important, such as with the use of electrodes, the adhesive sticker 30 preferably includes openings to allow for direct contact between the electrodes and the skin. Accordingly, as best seen in FIG. 3, the adhesive sticker 30 includes a pair of openings 31, 32 positioned to receive the anodes or other sensors 40, 41 formed in the casing of the patch.

Additional versions may include optical sensors in order to determine proximity. In such a version, a light sensors are positioned to detect light entering the bottom side of the patch; for example, one or more light sensors may be positioned in the location of one or both sensors 40, 41 as seen in FIG. 6. If the patch is properly applied to the head of the user, light would not be expected to enter and the patch would be determined to be properly adhered.

Yet other types of proximity sensors may be employed to detect whether the patch is attached to a head. For example, alternative sensors may take the form of temperature sensors configured to detect the temperature of the patch, taking into consideration an expected temperature range when the patch is in place atop a head. Still other sensors may monitor resistance, impedance, reactance, pressure or other parameters which may vary between conditions when the patch is worn or not worn by a user. Any of these or still other sensors may be used as proximity sensors.

In some versions of the invention, multiple proximity sensor types are used within a single patch. Thus, for example, a single patch may include one or more capacitive sensors together with one or more temperature sensors. One type or the other may be considered to be the primary or the backup form of sensor. Alternatively, the system may poll multiple sensors to determine that the patch is in position only if multiple sensors detect that it is in position.

As described above, the proximity sensor data may be used to prevent the operation of the impact sensors if the patch is not in position. Alternatively, it may allow the sensors to operate but the sensor module collects and pairs the data from the proximity sensors and the impact sensors to allow the system to determine which impact events are real and which are spurious. Either with the proximity sensors, or alternatively in the absence of the use of proximity sensors, the system may evaluate the impact sensor data to determine whether the patch was in position at the time of the impact event.

Several additional sensors may also be incorporated into the patch. One such sensor is a thermometer configured in position to detect the temperature at the patch. Most preferably, the thermometer is positioned sufficiently close to the adhesive portion of the patch (or through one of the openings in the sticker) such that the thermometer will detect the temperature of the wearer at the location of the patch. In one version, the detected temperature may be used to determine patch proximity and therefore whether the patch is in place on a user. In other versions, the thermometer data is collected and associated with impact sensor data to facilitate evaluation of the overall health of the wearer.

Further versions of the patch may include a heart rate sensor. As with temperature sensor, the heart rate sensor may be used to detect the presence of a pulse of the wearer and thereby confirm that the patch is positioned on a person. In addition, heart rate data may be collected by the patch and stored in the memory to track the user's heart rate, particularly at times before and after an impact event that may be detected by accelerometers or other such sensors.

An additional version of the patch may include a hydration sensor such as a low-cost, small microelectromechanical (MEMS) sensor that can be carried by the patch. The hydration sensor is positioned on the patch to make sufficient contact with the skin in order to detect the hydration of the wearer, preferably by being configured similarly as with the proximity sensors in order to extend through an opening in the sticker 30. Similarly, a sensor may include an electrolyte concentration sensor to detect and enable evaluation of the concentration of electrolytes in the user's system.

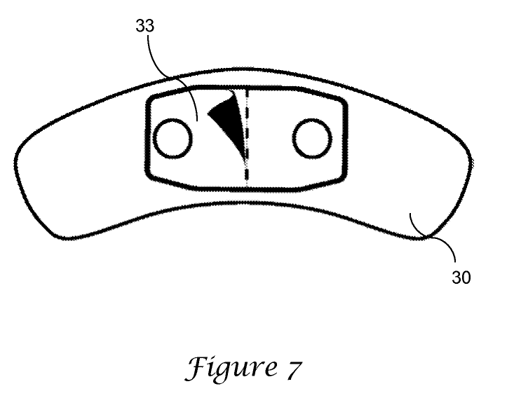

FIG. 7 illustrates an alternate preferred shape for a patch sticker 30. In this alternate version, the sticker is in the shape of a short arc, with the patch adhesion area 33 being positioned centrally on the arc. While the illustrated versions show the patch 20 as having a flat bottom, the patch may be formed with a bend or curvature that is expected to closely follow the contour of the occipital prominence where the sticker is preferably adhered. By forming the patch casing with such a curvature, it may result both in a more comfortable patch as applied, and cause the user to apply the patch in a desired manner, having a desired orientation.

As noted above, in some versions the patch is shaped in a manner in which the physical shape of the patch guides the user to apply it to the surface of the skin in a preferred orientation. Thus, where the patch is configured to have a shape that generally matches the region of exposed skin behind the ear, the wearer will have an increased probability of adhering the patch in a preferred orientation. In some versions, the orientation of the patch is guided by employing a patch adhesion area that matches that of the footprint of the patch, in which the footprint is asymmetrical or otherwise configured to ensure attachment to the patch in a predetermined orientation. In other versions, particularly where the patch is sufficiently miniaturized, the patch may be formed with a more symmetrical shape such as being round or oval. In such symmetrical versions (such as with the illustrated versions), the patch preferably includes an orientation sensor and/or indicator as described above in order to determine the orientation of the patch after it is applied.

As noted above, the patch preferably includes one or more sensors for proximity detection, as well as one or more additional sensors to detect parameters such as hydration, heart rate, or others. In some instances the sensors may require direct contact, such as in the case of electrodes employed as proximity sensors. In such instances, the adhesive is preferably applied in a manner to avoid interfering with the operation of the applicable sensors. In some cases, this may require that the adhesive not be applied in a manner that covers the sensors, while in other cases it may allow the sensors to be covered with a thin layer of adhesive.

The input-output interface is configured to allow data collected and processed by the patch to be transferred to another device for review and analysis, or to provide some measure of external feedback regarding the data obtained. In some versions, the input-output interface enables further computer programming instructions to be updated or otherwise transferred to the memory of the patch for operation by the processor.

In a simple form, the interface may be in the form of a connection point allowing for the removable connection of a wired interface to download data to a computer or other such device. For example, in a wired form the interface may allow for the connection of a wire having a USB connection for interfacing with a computer. In other versions, it may take the form of a transmitter or other wireless transmission means using Bluetooth, Wi-Fi or other formats. Most preferably, the interface allows for bidirectional communication, including the ability to download data and to perform onboard tasks such as reprogramming stored software or clearing data from memory.

In accordance with the preferred implementation of the patch as described above, the patch includes a processor and onboard memory. The memory contains stored programming instructions operable by the processor to perform a variety of functions as desired. In a simplest form, the memory simply stores the data as collected for evaluation at a later time, tracking data from each of the sensors and associating the data over time. In such a version, where the user does not experience any events worth subsequent evaluation, the data may be discarded. Alternatively, the data may be downloaded to a computer later for further analysis. The data analysis may take a variety of forms, and in many cases includes evaluating the accelerometer data to determine the nature and severity of an acceleration or impact event. In some instances this evaluation may further correlate the acceleration data with other sensor data such as heart rate, hydration, temperature, or other parameters as detected at the same points in time as the acceleration events.

While these evaluations may be performed on a computer after downloading the data, in a more complicated version they are performed on board by the processor. In some embodiments of versions employing onboard processing of this type, the input/output component may include the ability to sound an alarm through an onboard speaker, flash light for example through an onboard LED, or transmit wireless signals to a remote location using an onboard antenna to provide a similar form of audio, visual, or other notice that an event of note has occurred.

The data gathered by the sensors may further be used by the processor to determine a force vector experienced at the location of the patch. In one version, the processor may further translate the determined force vector to a different location within the head of the wearer, for example to a translated force vector representative of the force vector experienced by the center of mass of the head of the wearer. This process may, for example, be performed in accordance with the methods described in U.S. Pat. No. 8,466,794, the contents of which are incorporated by reference. While this process may be performed on-board the patch, it may alternatively be performed by a remote computer using the data output by the patch.

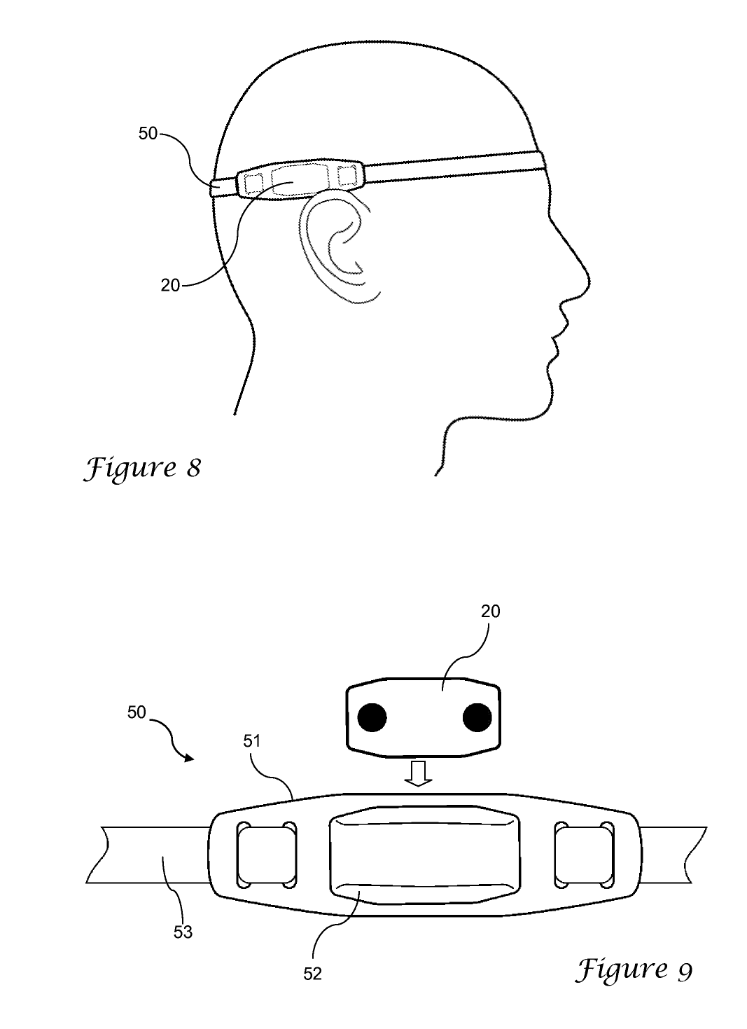

An alternate version of the patch 20 may be incorporated into a headband 50, as best seen with reference to FIGS. 8 and 9. In this version, the patch 20 is preferably configured as illustrated and described above with reference to FIGS. 1-6, differing primarily in that the headband version preferably does not include a sticker for adhesion to a wearer, and instead uses a headband 50 to attach the patch 20 to a wearer.

In one preferred headband version, the headband 50 includes a strap 53 configured to encircle the wearer's head. In some versions the strap includes a feature allowing for it to be adjusted, such as a buckle or a hook and loop fastener. The strap may also be formed from an elastic material allowing for a single strap to fit heads of varying sizes. The strap supports a sheath 51 forming a seat 52 for receiving and retaining the patch 20. In a preferred version, the seat and casing of the patch are formed in a complementary fashion to allow for a snap-fit, friction-fit, or similar method of attachment that allows the patch to be firmly held within the sheath but removable when desired. When the patch is positioned within the sheath, the headband may be placed about the head of a wearer.

In accordance with the features described above, the patch for use with the headband is preferably configured in the manner as described above, incorporating sensors and other components as described with reference to FIG. 2, as well as proximity sensors and orientation indicators as described above. As illustrated in FIG. 8, a headband sensor is preferably positioned above and just behind the ear, and therefore the casing preferably includes a position indicator pointing to the ear as illustrated in FIG. 5.

A patch as described above may be used for a variety of purposes. One example, the patch may be used by athletes playing football, soccer, basketball, boxing, or other sports in order to track instances in which the head experienced an impact that might be associated with a concussion or other injury or condition. Where such an event has occurred, additional sensors on the patch will have collected additional information stored in memory and accessible to be associated with the timing of the impact event in order to better evaluate the health of the wearer by, for example, reviewing the temperature, hydration, heart rate, or other health parameters before, during, and after the event.

The patch may also be used in other settings, for example to detect fatigue or distraction. This feature may be useful for drivers, security guards, pilots, or other personnel in positions where it is important to stay awake and avoid distraction. In such a setting, the memory on board the patch may be programmed with instructions operable by the processor to look for particular patterns of head movement. For example, a drowsy driver may typically nod for a period of time, thereby indicating that the driver is having difficulty staying awake and is about to fall asleep at the wheel. Thus, the stored programming instructions will evaluate head movements in order to detect up and down movements over short periods of time associated with nodding. In the event the wearer is nodding in a pattern associated with drowsiness, the processor may cause an onboard alarm to sound, or may send a wireless signal to a remote alarm, thereby causing the driver to become more alert and aware of the drowsiness situation.

Alternatively, the processor may be programmed to evaluate head position in order to determine whether the wearer is looking straight ahead or, alternatively, positioned with the head downward and indicating that the chin is positioned toward the chest and the wearer is asleep or inattentive. Similarly, a steeply inclined angle in which the head may be interpreted as being tipped back in a sleeping position may be determined. As with the version above, the processor may be programmed to sound an alarm, send a signal, or flash light in such a situation.

The patch may further be used by personnel in any hazardous situation, such as by soldiers in battle, in order to maintain data regarding the health of the wearer. In the event a soldier is injured or otherwise incapacitated, the data collected by the patch may be accessed in order to provide additional information regarding the condition of the wearer at the time of the incapacitation. For example, it may indicate that the wearer experienced an impact event to the head and then lost consciousness. Alternatively, it may indicate a drop in heart rate or dehydration perhaps suggesting that the wearer lost consciousness by fainting or some other condition rather than a blow to the head. In either case, the stored information collected in the memory may be used by a first responder or healthcare professional to better evaluate the health of the wearer.

While the preferred embodiment of the invention has been illustrated and described, as noted above, many changes can be made without departing from the spirit and scope of the invention. Accordingly, the scope of the invention is not limited by the disclosure of the preferred embodiment. Instead, the invention should be determined entirely by reference to the claims that follow.

The embodiments of the invention in which an exclusive property or privilege is claimed are defined as follows:

1. An adhesive shock patch, comprising:
an impact sensor configured to sense an impact parameter experienced by the patch;
a processor;
a memory in communication with the processor, the memory containing stored programming instructions operable by the processor to receive and store sensor data from the impact sensor; and
an adhesive applied to the patch to adhere the patch to a region of skin on a user.

2. The adhesive shock patch of claim 1, further comprising a casing, the impact sensor, processor and memory being housed within the casing.

3. The adhesive shock patch of claim 2, further comprising a sticker removably attachable to the casing, the sticker having a front side and a back side, wherein the adhesive applied to the patch comprises an adhesive applied to a back side of the sticker, the casing and front side of the sticker further each having one of a complementary portion of a hook and loop fastener for removably attaching the casing to the front side of the sticker.

4. The adhesive shock patch of claim 2, further comprising a sticker removably attachable to the casing, the sticker having a front side and a back side, wherein the adhesive applied to the patch comprises an adhesive applied to a back side of the sticker, the casing being removably attached to the front side of the sticker.

5. The adhesive shock patch of claim 4, further comprising a proximity sensor extending through the casing, the sticker further having a hole formed in the sticker at a location to facilitate interaction between the proximity sensor and the skin of the user.

6. The adhesive shock patch of claim 5, wherein the proximity sensor comprises a pair of electrodes extending through the casing, the hole in the sticker further comprising a pair of holes positioned to receive the pair of electrodes.

7. The adhesive shock patch of claim 5, wherein the sticker comprises an outer sticker perimeter and the casing comprises an outer casing perimeter, the sticker perimeter extending beyond the casing perimeter when the casing is attached to the sticker.

8. The adhesive shock patch of claim 5, wherein the casing further comprises an orientation indicator.

9. The adhesive shock patch of claim 8, wherein the orientation indicator comprises an indicator pointing to a direction of an ear of the user when the patch is attached to the user in a predetermined orientation.

10. The adhesive shock patch of claim 9, wherein the sticker forms a pear shape.

11. The adhesive shock patch of claim 5, wherein the stored programming instructions further comprise instructions interpret data from the impact sensor to determine an axial orientation of the patch.

12. The adhesive shock patch of claim 5, wherein the impact sensors comprise a plurality of accelerometers.

13. The adhesive shock patch of claim 5, wherein the impact sensors comprise a plurality of gyroscopes.

14. The adhesive shock patch of claim 5, further comprising a wireless transmitter configured to wirelessly transmit the sensor data.

15. The adhesive shock patch of claim 5, further comprising an input/output interface, the input/output interface being configured to enable transfer of the sensor data to a remote computer and to enable transfer of further programming instructions to the memory for operation by the processor.

16. An adhesive shock patch, comprising:
a casing, the casing housing:
(1) an impact sensor configured to sense an impact parameter experienced by the patch;
(2) a processor; and
(3) a memory in communication with the processor, the memory containing stored programming instructions operable by the processor to receive and store sensor data from the impact sensor; and
a sticker removably attached to the casing, the sticker having an adhesive to removably adhere the patch to a region of skin on a user.

17. The adhesive shock patch of claim 16, further comprising a proximity sensor extending through the casing, the sticker further having a hole formed in the sticker at a location to facilitate interaction between the proximity sensor and the skin of the user.

18. The adhesive shock patch of claim 17, wherein the casing is formed from a rigid material.

19. The adhesive shock patch of claim 17, wherein the stored programming instructions further comprise instructions for determining an axial orientation of the patch.

20. The adhesive shock patch of claim 17, wherein the casing further comprises an orientation indicator.
